(19) United States
(12) Patent Application Publication
(10) Pub. No.: US 2011/0031060 A1
(43) Pub. Date: Feb. 10, 2011

(54) Earpiece

(75) Inventors: Heinz Epping, Hildesheim (DE); Dirk Hackbarth, Hannover (DE); Markus Kuhr, Singapore (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sennheiser electronic GmbH & Co., KG, Wedemark (DE)

(21) Appl. No.: 12/745,471

(22) PCT Filed: Dec. 3, 2008
(86) PCT No.: PCT/EP08/66685

§ 371 Date: Oct. 25, 2010

(30) Foreign Application Priority Data
Dec. 3, 2007 (DE) 20 2007 016 881.0

(51) Int. Cl. A61F 11/08

(52) U.S. Cl. 181/135

(57) ABSTRACT

There is provided an earpiece having at least one housing (2) having a first end for receiving an earplug (1) and a second end for receiving a cap unit (3). In that case the cap unit is adapted to be removable and/or movable relative to the housing.

The invention concerns an earpiece.

Hitherto earpieces are known in different configurations. By virtue of their structural shape they can be distinguished as in-ear earpieces and on-ear earpieces. In-ear earpieces are introduced into the auditory canal and therefore already afford a certain degree of insulation in relation to external noises. On-ear earpieces in contrast are placed over the outer ear and in that case either rest against it or totally enclose the ear. In that respect on-ear earpieces can either be closed or (semi-) open. With the closed configuration a greater bass pressure is achieved, which in the (semi-) open configuration has to be achieved by the technology of sound generation. Sound generation involves using very substantially electrodynamic systems which have superseded electromagnetic technologies. In addition electrostatic and also piezoelectric systems are possible.

The acoustic performance of the earpiece is substantially determined by the combination of the structural configuration with the technology of sound generation. In that respect there are far-reaching possible ways in respect of sound generation for establishing the acoustic performance by the implementation of acoustic transducer technology upon production and further influencing options by virtue of actuation of the acoustic transducer. Furthermore the structural configuration of the earpiece influences sound transmission and propagation. In that respect both in in-ear earpieces and also in on-ear earpieces the acoustic transducer is surrounded by a housing which is capable of transmitting the sound on the side of the auditory canal and permits propagation of the sound in the direction of the eardrum. The housing seals off hearing relative to the exterior around the acoustic transducer, in which case the design configuration in the form of an in-ear earpiece or an on-ear earpiece manage that task by virtue of different configurations. In an outward direction the housing in each case is of such a configuration that the propagation of sound in that direction is avoided as much as possible. Thus a defined volume is available within the housing, which volume can be excited by the acoustic transducer and the sound waves of which can be propagated as far as possible exclusively in the direction of the eardrum. In that respect the nature and quality of the sound waves is established by the totality of the mechanical properties of the earpiece and the acoustic transducer technology employed.

What is common to all the above-described earpieces in that respect is that their acoustic properties are very substantially established upon manufacture thereof. Therefore actuation of the acoustic transducer generally represents the sole possible way of influencing the properties of the acoustic sound generated, by changes in the electrical signal. Accordingly those influencing options are displaced away from the earpiece to the signal source of the electrical signal. If in contrast the acoustic properties can be also altered at the acoustic transducer of the earpiece, such an option is to be provided in the electronic system of the acoustic transducer, whereby a corresponding increase in complication and expense has to be involved in development and manufacture and the structural size of the acoustic transducer can also be increased.

U.S. No. 2007/0177753 A1 discloses an in-ear earpiece having a passage for connecting the internal volume delimited by the housing of the earpiece, to an external volume. Provided at the exit of the passage is a closure unit for closing or opening the passage. The closure can be closed or opened for example by means of a displaceable cover.

Therefore the object of the invention is to provide an easier possible way of influencing the acoustic performance of an earpiece.

That object is attained by an earpiece as set forth in claim 1.

Thus there is provided an earpiece comprising at least one housing having a first end for receiving an earplug and a second end for receiving a cap unit. In that case the cap unit is adapted to be removable and/or movable relative to the housing.

An advantage of the earpiece according to the invention is that the housing of the earpiece at its second end is of such a configuration that a cap unit can be received there. That cap unit is held at that end, but it is removable.

That provides that the acoustic properties of the earpiece are not definitively established upon production, but can be adapted during use by the user to his individual sound ideas and requirements. That is effected by alterations to the housing which in accordance with the invention is so designed that its action on the acoustic performance of the earphone can be altered by simple mechanical alterations.

In accordance with an aspect of the invention the cap unit can be both removed and also fixed in place without the use of tools. That makes it possible for the user to be able to carry out alterations to the housing of the earpiece very quickly, easily and comfortably himself.

In accordance with a further aspect of the invention different cap units are of such a configuration that they have a different acoustic performance. In that way it is possible to provide for a different acoustic performance on the part of the earphone by the removal of a cap unit and fixing another cap unit in place.

In accordance with an aspect of the invention different cap units can include different volumes. As the acoustic performance of the earpiece is influenced by the volume enclosed by the earpiece housing the acoustic performance of the overall earpiece can be specifically and targetedly influenced by way of the enclosed volume of the cap unit.

In accordance with an aspect of the invention the cap units have at least one opening. As the sound which is propagated in the enclosed volume can also be propagated outwardly therethrough, the acoustic performance of the overall earpiece can also be specifically and targetedly influenced by the nature and number of the openings in the cap units.

In accordance with an aspect of the invention acoustic resistance material or different acoustic resistance materials is or are provided at least in or at an opening. The effect of the opening on the acoustic performance of the earpiece can further be specifically and targetedly influenced by the nature of the material.

The invention also concerns an earpiece in which the housing of the earpiece at its second end is of such a configuration that the cap unit is movable with respect to the housing. That means that the acoustic properties of the earpiece are also not definitively fixed upon production but can be adapted during use by the user to his individual sound ideas and requirements. That is effected solely by movement of the cap unit with respect to the housing which in accordance with the invention is of such a configuration that the acoustic performance of the earpiece can be altered by the position of the cap unit and the housing relative to each other.

In that respect it is advantageous if the cap unit and the housing each have at least one opening which are disposed in the same plane. In that way the openings of the cap unit and the housing can be brought into coincidence with each other by the movement of the cap unit and the housing relative to each other. That means that the earpiece has a different adjustable acoustic performance, depending on whether the two openings are or are not in alignment.

In accordance with an aspect of the invention an acoustic resistance material or different acoustic resistance materials is or are provided in or at at least one opening in the cap unit and/or the housing. The effect of the opening on the acoustic performance of the earpiece can further be specifically and targetedly influenced by the nature of the material.

In accordance with an aspect of the invention the openings in the cap unit and/or housing are of such a configuration that the openings can be at least partially brought into coincidence by the movement of the cap unit with respect to the housing. That provides that the resulting opening which acts on the acoustic performance of the earpiece corresponds to the mutually coincident openings of the cap unit and the housing. That permits increasing overlapping coincidence of the corresponding openings with a corresponding action on the acoustic performance of the earpiece.

In accordance with an aspect of the invention the cap unit which is movable with respect to the housing can be both removed and also fixed in place without the use of tools. That makes it possible for the user to be able to effect alterations to the housing of the earpiece very quickly, simply and conveniently himself.

In accordance with an aspect of the invention different cap units which are movable with respect to the housing can enclose different volumes. As the acoustic performance of the earpiece is influenced by the volume enclosed by the earpiece housing the acoustic performance of the overall earpiece can be specifically and targetedly influenced by way of the enclosed volume of the cap unit.

Embodiments by way of example and advantages of the invention are described in greater detail hereinafter with reference to the following Figures:

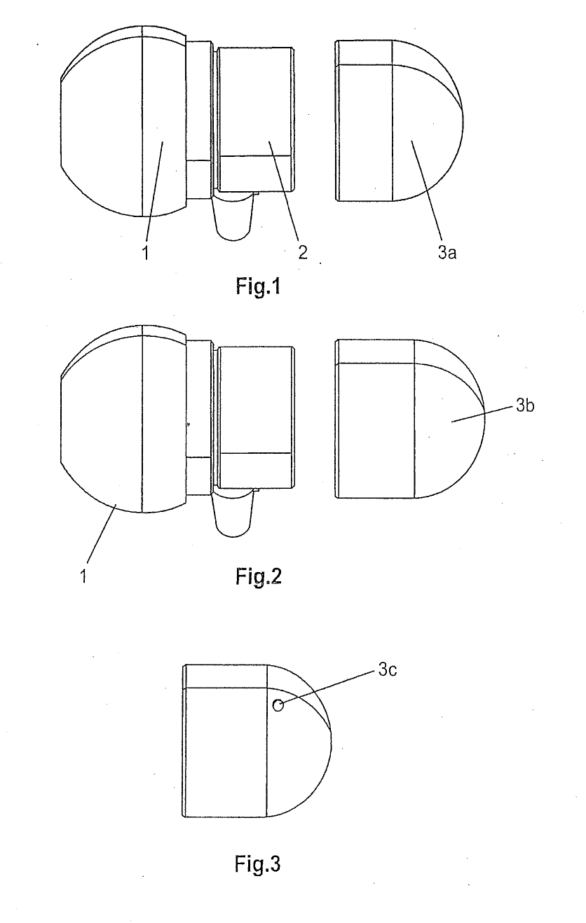

FIG. 1 shows a plan view of an earpiece in accordance with a first embodiment,

FIG. 2 shows a further plan view of an earpiece in accordance with a first embodiment,

FIG. 3 shows a plan view of a cap in accordance with the first embodiment,

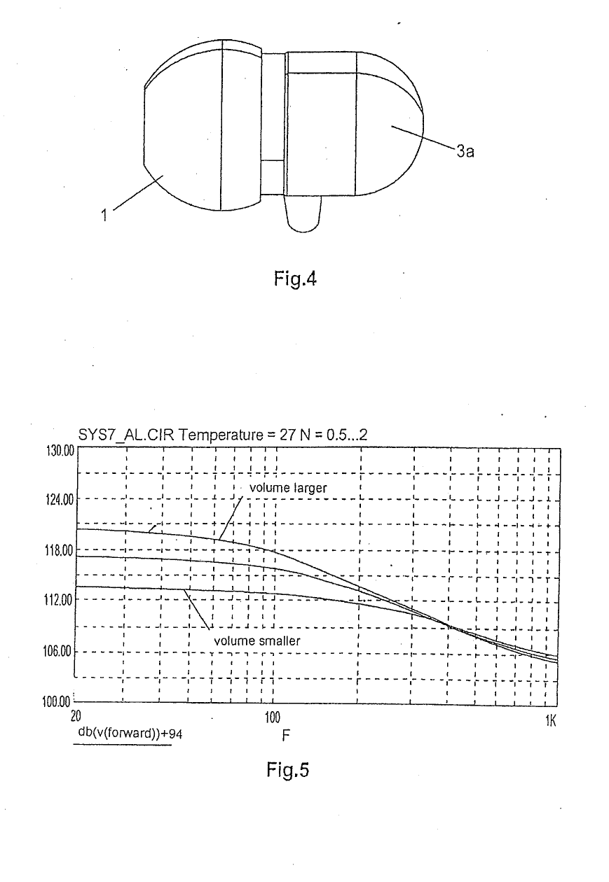

FIG. 4 FIG. 2 shows a further plan view of an earpiece in accordance with a first embodiment,

FIG. 5 shows a frequency characteristic of the earpiece in accordance with the first embodiment,

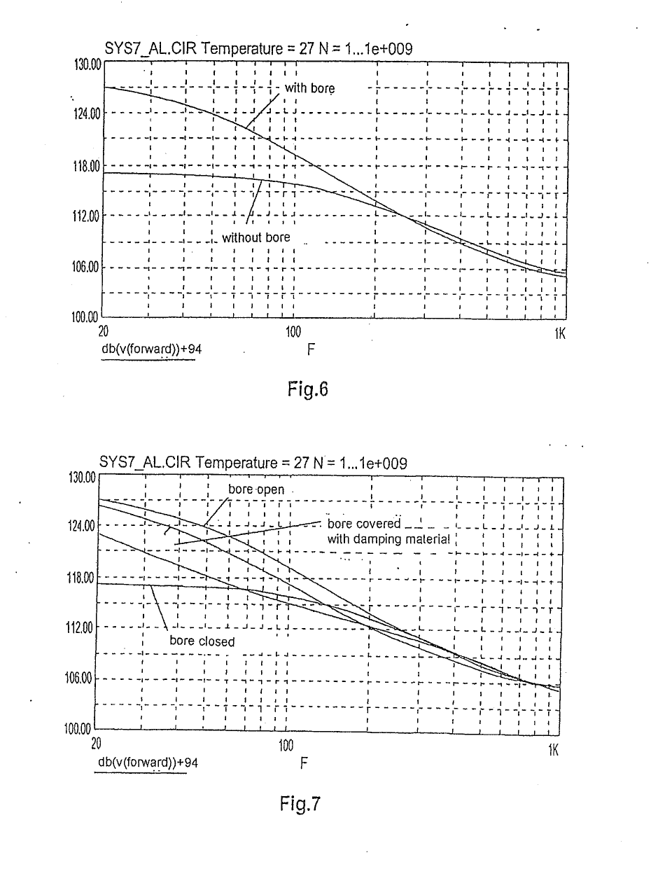

FIG. 6 shows a further frequency characteristic of the earpiece in accordance with a first embodiment,

FIG. 7 shows a further frequency characteristic of the earpiece in accordance with the first embodiment,

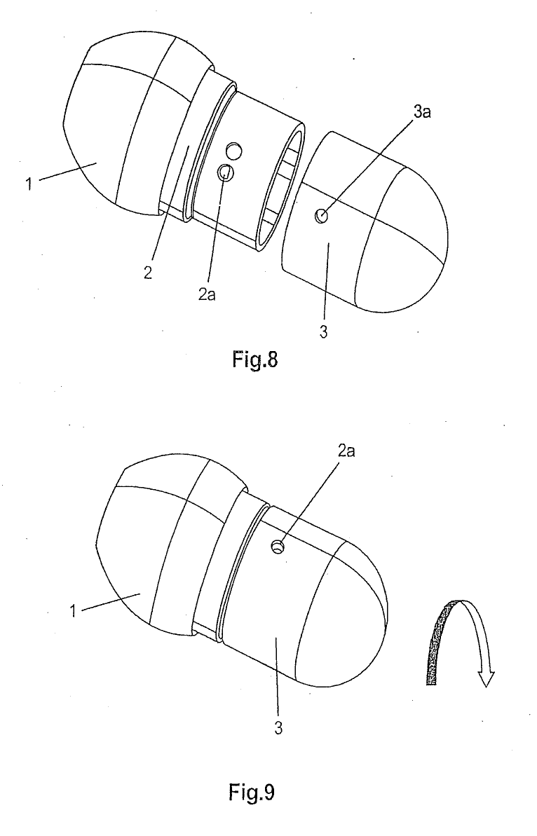

FIG. 8 shows a plan view of an earpiece in accordance with a second embodiment,

FIG. 9 shows a further plan view of an earpiece in accordance with the second embodiment,

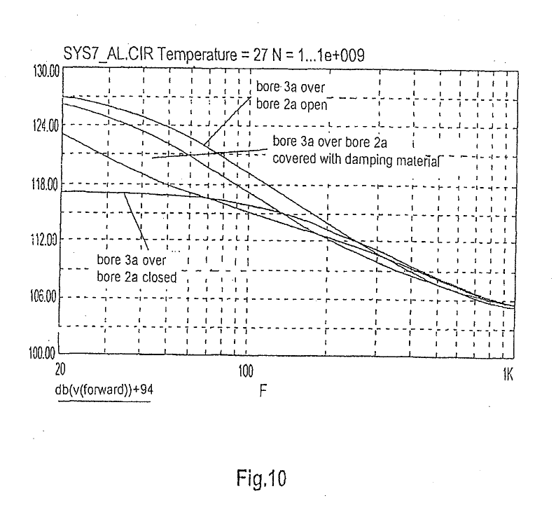

FIG. 10 shows a frequency characteristic of the earpiece in accordance with the second embodiment,

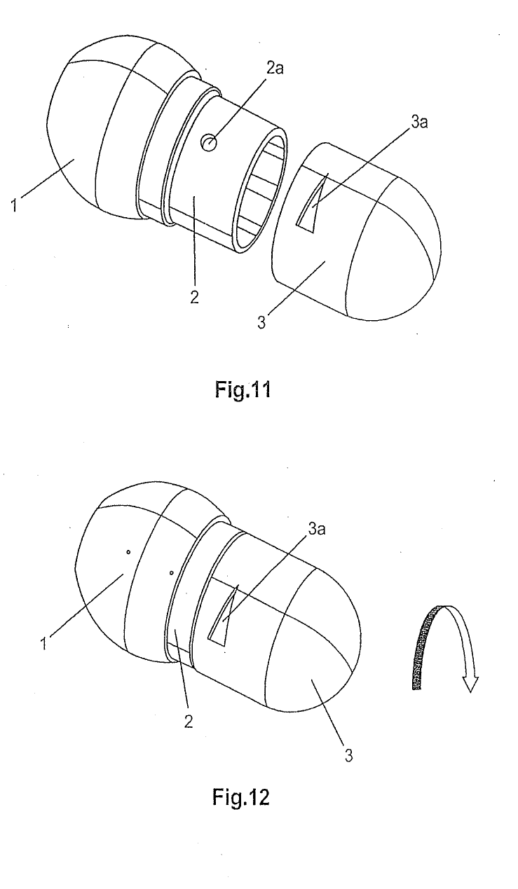

FIG. 11 shows a further plan view of an earpiece in accordance with the second embodiment,

FIG. 12 shows a further plan view of an earpiece in accordance with the second embodiment,

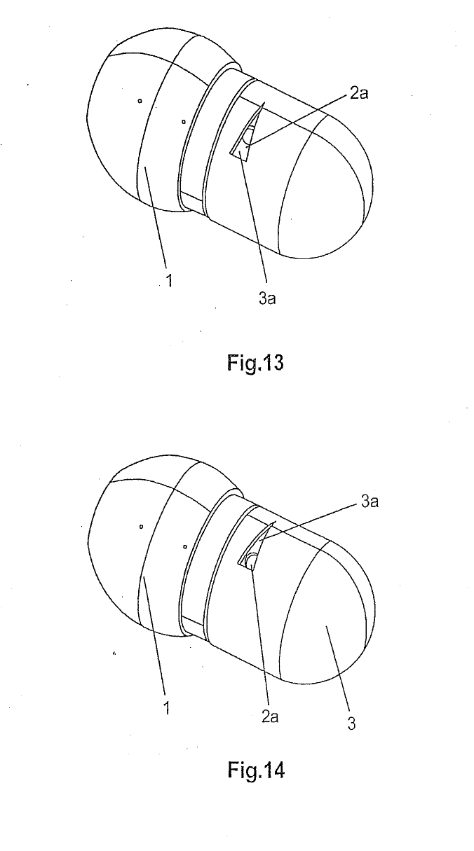

FIG. 13 shows a further plan view of an earpiece in accordance with the second embodiment,

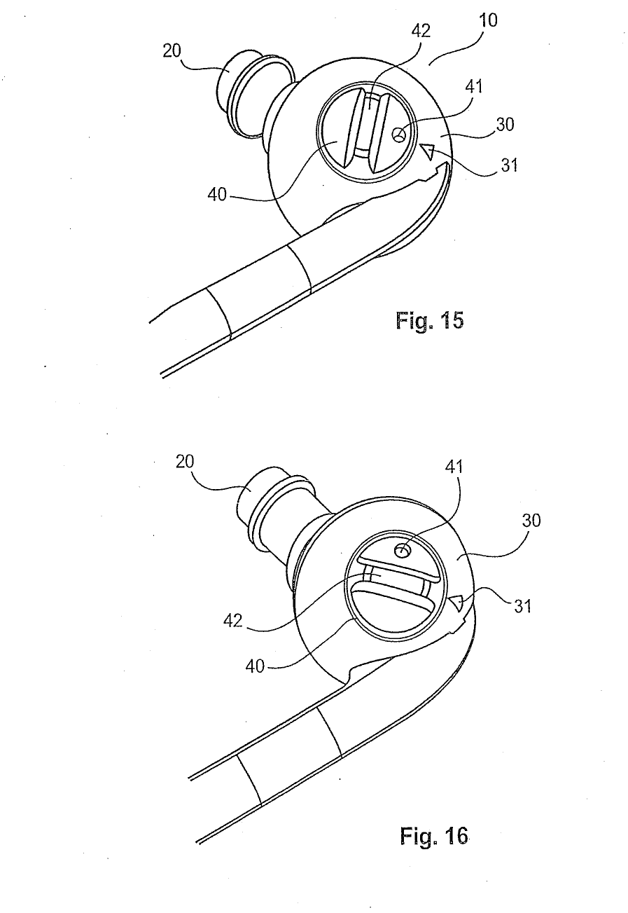

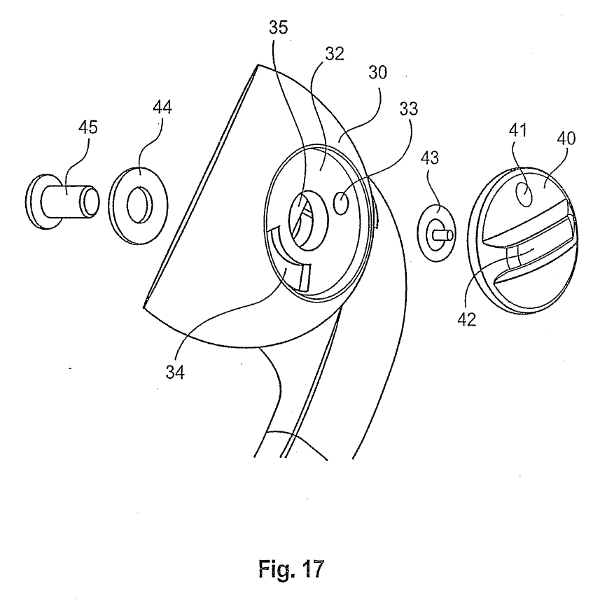

FIG. 14 shows a further plan view of an earpiece in accordance with the second embodiment,

FIG. 15 shows a diagrammatic view of an in-ear earpiece in accordance with a third embodiment,

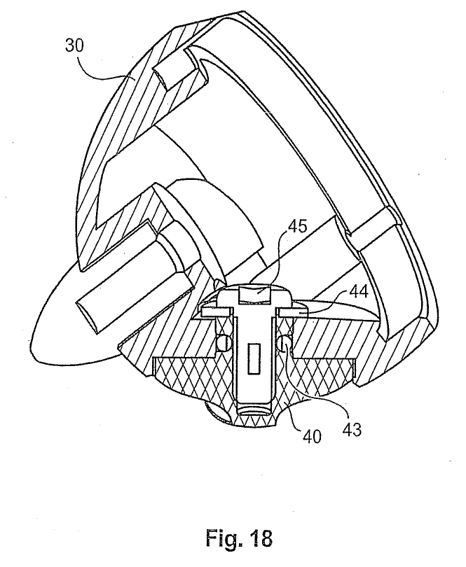

FIG. 16 shows a further diagrammatic view of the in-ear earpiece in accordance with the third embodiment,

FIG. 17 shows a diagrammatic view of an in-ear earpiece in accordance with the third embodiment,

FIG. 18 shows a diagrammatic sectional view of the second end of the housing of the in-ear earpiece in accordance with the third embodiment, and

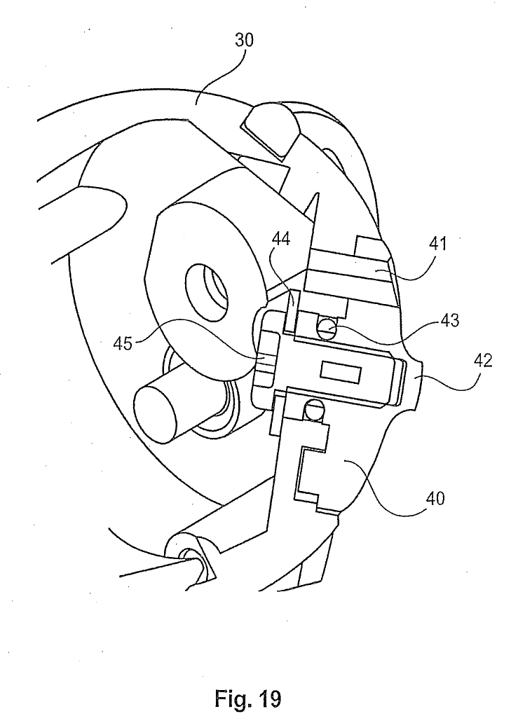

FIG. 19 shows a further diagrammatic sectional view of the housing of the in-ear earpiece in accordance with the third embodiment.

FIG. 1 shows an in-ear earpiece or earphone comprising a housing 2 which at its first end has or can receive an earplug 1 and at its second end has or can receive a cap unit 3. In this case the earplug 1 can serve to promote the delivery of sound into the auditory canal of the user. In addition the earplug 1 serves for improved wearing comfort for the earpiece. The earplug 1 is generally made from soft materials which afford a pleasant wearing comfort for the user and provide for well isolating the auditory canal in relation to external noises. Disposed in the housing 2 itself is an acoustic transducer which operates in accordance with the electrodynamic or electrostatic principle. That involves a diaphragm being caused to oscillate so that sound waves are propagated from that diaphragm into the surrounding space. As the sound is propagated in all directions in space, it is necessary for the housing 2 of the earpiece to be at least partially closed off outwardly by a cap unit 3. In that way, the housing 2, the cap unit 3 and the auditory canal of the user form a closed volume in which the sound can be propagated in the direction of the eardrum of the user. In that respect both the size of the enclosed volume and also the acoustic isolation of that volume in an outward direction have an influence on the acoustic performance of the earpiece.

Hitherto the mechanical properties of an earpiece are definitively established upon manufacture thereof by the housing of the earpiece being provided in the form of a closed unit. That means that the enclosed volume within the housing is also fixed and therewith also the acoustic influences resulting from the mechanical nature of the earpiece. Therefore, only the electroacoustic sound transducer effect is available for influencing the sound characteristics of the earpiece. The electroacoustic sound transducer effect can be influenced by control of the electrical signals. In addition, upon manufacture the acoustic transducer and its electronics can also be designed for example by the use of switchable resistors in such a way that there are further possible options for altering the sound characteristics. It will be noted however that this requires additional complication and expenditure in manufacture. In addition further components are required and the structural size of the acoustic transducer can be increased. Finally, that also fixes the possible options in terms of altering the acoustic performance upon manufacture.

In contrast thereto the invention provides inter alia a simple mechanical possible way of being able to adapt the acoustic performance of the earpiece by the user to the user ideas and requirements. For that purpose in accordance with the invention the housing 2 of the earpiece is of such a configuration that the housing 2 is closed off outwardly by a cap unit 3. The nature of that cap unit makes it possible for the size of the enclosed volume as well as acoustic isolation of that volume in an outward direction to be also specifically and targetedly influenced after manufacture.

In a first embodiment that influencing aspect is afforded by virtue of the fact that the cap unit 3 can be removed from the housing 2 or partially pulled off and fixed thereto. In that respect it is advantageous if removal and fixing can be effected by the user without the use of a tool, that is to say in a tool-free fashion. That permits fast simple replacement of the cap unit 3, by the user.

FIGS. 2 and 3 each show a further plan view of an earpiece in accordance with the first embodiment. To be able to achieve different acoustic characteristics for the earpiece by replacement of the cap unit 3, different cap units 3a, 3b and 3c can have or cause different acoustic properties or acoustic volumes. That provides for making available to the user a selection of cap units which he can fix to the housing 2 and also remove again without the use of a tool, depending on his respective individual ideas and requirements. As a result the acoustic performance of the earpiece is not definitively established upon manufacture, but can be altered within the limits of the existing interchangeable cap units.

In that respect, the volume which is enclosed by the housing 2. the cap unit 3 and the auditory canal of the user is available for influencing the sound characteristics, by virtue of the nature of the cap unit 3. Therefore the cap units 3a and 3b are to be so provided that they are of a differing size and thereby enclose a differing volume.

In addition the isolation of the earpiece in relation to the environment can be specifically and targetedly influenced by the nature of the cap unit 3. Therefore at least one opening can be provided in the cap unit 3c (FIG. 3), the opening permitting the sound to escape outwardly through the cap unit 3c in a defined fashion. In that respect the escape of sound can be established by the size, nature and number of the openings in the cap unit 3c. Different settings for the escape of sound can be achieved by virtue of different cap units 3c provided with openings.

A further possible way of influencing the propagation of sound outwardly involves providing acoustic resistance material in or at the openings 3c. That offers a multiplicity of possible design options as the cap units 3c can have one or more openings, in respect of which an acoustic resistance material can be provided at no, one or also a plurality of openings, completely or also only partially. The same or different materials can also be provided at a plurality of openings. That makes it possible to provide a wide bandwidth in respect of cap units 3c for the user.

FIG. 4 shows a plan view of an earpiece in accordance with the first embodiment. FIG. 4 shows how a cap is fitted on an end of the earpiece.

FIGS. 5 through 7 respectively show a frequency characteristic of an earpiece in accordance with the first embodiment with different caps. Thus, by selecting the cap which is placed on the earpiece, it is possible to select which low-frequency characteristics of the earpiece are to be set. In the case of the earpiece in accordance with the first embodiment it is possible to adjust in particular the low-frequency characteristic.

In the second embodiment the acoustic characteristics of the earpiece are influenced in accordance with the invention by the cap unit 3 being adapted to be movable relative to the housing 2. That affords the user a further possible way of being able to quickly and easily alter the acoustic characteristics of the earpiece by way of the position of the cap unit 3 relative to the housing 2.

FIGS. 8 and 9 respectively show a plan view of an earpiece in accordance with the second embodiment. Both the housing 2 and also the cap unit 3 may each have at least one opening 2a and 3a, see FIG. 8. Those openings 2a, 3a can be disposed in one plane so that they can be brought into coincident relationship with each other for example by rotation of the cap unit 3 relative to the housing 2. If at least two openings 2a and 3a are in coincident relationship with each other, that results in an opening of the earpiece in relation to the outside space so that sound can issue through that resulting opening. That affords specifically targeted influencing of the acoustic characteristics of the earpiece.

With those openings 2a and 3a it is also possible to provide acoustic resistance material in or at the openings 2a and/or 3a. That also provides in the second embodiment numerous possible design options for the acoustic characteristics of the earpiece as one or more openings can be provided in the cap unit 3 and the housing 2, in respect of which an acoustic resistance material can be provided at no, one or also a plurality of openings completely or also only partially. The same or also different materials can also be provided at a plurality of openings, see FIG. 8.

FIG. 10 shows a frequency characteristic of the earpiece in accordance with the second embodiment. The graph in FIG. 10 shows a curve for the earpiece in accordance with the second embodiment when the bore 3a is over the bore 2a and the bores are thus open. Also shown is a frequency characteristic at which the bore 3a over the bore 2a is covered with damping material. Finally a frequency characteristic is also shown for an earpiece, in which the bore 3a over the bore 2a is closed.

FIGS. 11 through 14 each show a further plan view of an earpiece in accordance with the second embodiment. The configuration of the openings 2a and 3a is of particular significance in the second embodiment. As sound can issue outwardly only through the resulting opening which results from the openings 2a and 3a in the cap unit 3 and the housing 2 being in coincident relationship, the nature of coincidence of the openings 2a and 3a offers a great scope for handling in terms of influencing the acoustic characteristics. Thus only one single opening 2a or 3a can be brought completely into coincident relationship with a corresponding opening in the counterpart 2a or 3a, see FIGS. 8 and 9. A plurality of openings 2a or 3a can also be brought into coincident relationship with only one suitably large opening in the counterpart 2a and 3a. The configuration of openings 2a and/or 3a is also possible, which by virtue of their shape permit increasing overlap of one or more openings 2a and 3a, see FIGS. 11 through 14. In that respect the shape of the openings 2a and/or 3a may be such that it is possible to set both stepless and also stepped overlap of the openings 2a and 3a.

A further embodiment represents a cap unit whose enclosed volume can be adjusted by movement of the cap unit and the housing relative to each other. In that case the cap unit is to be of such a design that it can be moved and fixed with respect to the housing without a tool. In that way the volume enclosed by the housing and the cap unit can be altered in steps not only by replacement of the cap unit, but the cap unit respectively fixed to the housing can be displaced by movement with respect to the housing. The volume enclosed by the housing and the cap unit can be steplessly varied in that case. In addition it is also possible to use any kinds of openings, as described for the first two embodiments, on this third embodiment.

The described embodiments of the invention are particularly suitable for use in relation to in-ear earpieces. It will be noted however that the use of earpieces according to the invention is also possible in relation to other earpieces, such as for example on-ear earpieces.

The invention concerns the concept of providing an in-ear earpiece to which a plurality of acoustically tuned components are added. Those components can be fixed to the earpiece or removed again by the customer without a tool. In that way the customer can adjust for example the low-frequency characteristic of the earpiece in accordance with his sound requirements.

The invention further concerns the idea of providing an in-ear earpiece whose housing cap can be rotated about the longitudinal axis. Depending on the respective position of the cap for example the low-frequency characteristic changes. That means that the customer can adjust the low-frequency characteristic of the earpiece in accordance with his sound requirements.

FIG. 15 shows a diagrammatic view of an in-ear earpiece in accordance with a third embodiment. The in-ear earpiece in accordance with the third embodiment has a first end 20 for receiving an earplug and a second end 30. Provided at the second end 30 is a movable or rotatable operating unit or (closure) cap 40 having a hole 41. The operating unit or cap 40 is adapted to be rotatable.

FIG. 16 shows a further diagrammatic view of the in-ear earpiece in accordance with the third embodiment. In this case the in-ear earpiece shown in FIG. 16 corresponds to the in-ear earpiece shown in FIG. 15. The only difference between those two Figures is that the movable or rotatable operating unit or cap 40 has been rotated in the counter-clockwise direction so that the hole 41 now points upwardly.

Preferably provided in the housing 30 is a hole or an opening and that hole or opening can be closed or opened by rotation of the operating element or cap 40. When the hole 41 in the operating element or cap 40 is brought into conformity with a hole in the second end 30 of the housing by rotation of the operating element or cap 40, the internal volume of the housing is then connected to the external volume.

In other words, by means of the operating element or the cap, an opening or a hole in the housing of the in-ear earpiece can be opened or closed from the outside by the user.

FIG. 17 shows a diagrammatic view of an in-ear earpiece in accordance with the third embodiment. FIG. 17 shows in particular the second end 30 of the housing and further elements of the cap 40. Provided in the second end 30 of the housing are a first hole 33 which is in the form of an acoustically tunable hole and a second larger hole 35 for receiving an end of the cap 40. In that case the first and second holes are preferably provided in a recess 32 at the second end 30. In addition it is possible to provide in the recess 32 an opening 33 which can preferably represent an elongated or elongate opening or a slot. The operating element or the closure unit or closure cap 40 can be positioned in the recess 32 and fixed for example by means of a screw 45 and a nut 40 from the inside region of the second end 30 of the housing. It is also possible to provide an O-ring 43 (for example of silicone) which can be provided at an end of the cap 40.

FIG. 18 shows a diagrammatic sectional view of the second end of the housing of the in-ear earpiece in accordance with the third embodiment. FIG. 18 shows the operating element 40 in the installed condition, that is to say the screw 45 and the nut 44 are connected or screwed from the inside of the second end 30 of the housing to the operating element or the cap 40.

FIG. 19 shows a further diagrammatic sectional view of the housing of the in-ear earpiece in accordance with the third embodiment. Here the operating element 40 is also fixed by means of the screw 45 and the nut 44 to the second end 30 of the housing. The cap or the operating element 40 has a through hole 41.

In this respect the above-described fixing of the operating element 40 is preferably effected in such a way that the operating element 40 can be rotated in order to bring the hole 33 in the second end 30 into coincident relationship with the (through) hole 41 in the operating element or to close the hole 33.

On the inside of the operating unit 40 or the cap unit 40 there can be provided a nose 46 which can co-operate with the slot 34 when the cap unit 40 is placed in the recess 32. The co-operation between the nose and the slot means that it is possible to provide two limitations in respect of the position of the cap unit (for example closed or open).

The earpiece or in-ear earpiece in accordance with the third embodiment preferably has an opening 35 for receiving the cap 40 in its rear volume, that is to say behind the electroacoustic playback transducer.

1. An earpiece comprising:
at least one housing having a first end for receiving an earplug and a second end for receiving a cap unit adapted to be removable.

2. An earpiece as set forth in claim 1 characterised in that the cap unit can be removed and fixed without a tool.

3. An earpiece as set forth in claim 1 characterised in that the second end can receive different cap units, wherein the different cap units have different acoustic properties.

4. An earpiece as set forth in claim 1, characterised in that the second end can receive different cap units, wherein the different cap units enclose different volumes.

5. An earpiece as set forth in claim 1 characterised in that the cap unit has at least one opening.

6. An earpiece as set forth in claim 1 characterised in that an acoustic resistance material is provided in or at least one opening of the cap unit.

7. An earpiece comprising:
at least one housing having a first end for receiving an earplug and a second end for receiving a cap unit;
wherein the cap unit is adapted to be movable relative to the housing.

8. An earpiece as set forth in claim 7 characterised in that the cap unit and the housing each have at least one respective opening which are disposed in the same plane.

9. An earpiece as set forth in claim 7, wherein the cap unit has a respective opening and the housing has a respective opening, the earpiece being characterised in that the openings of the cap unit and the housing can be at least partially brought into coincident relationship with each other by displacement relative to each other.

10. An earpiece as set forth in claim 7 characterised in that an acoustic resistance material is provided in or at least one opening of the cap unit or the housing.

11. An earpiece as set forth in claim 7 characterised in that the cap unit can be removed and fixed without a tool.

12. An earpiece as set forth in claim 7 characterised in that the second end can receive different cap units, wherein the different cap units enclose different volumes.

13. An earpiece as set forth in claim 1 characterised in that the earpiece is in the form of an in-ear earpiece.

14. An earpiece, comprising an in-ear earpiece, comprising:
at least one housing having a first end for receiving an earplug, and
a second end having at least one hole or opening,
wherein provided at the second end is a rotatable cap unit for closing and opening the hole or opening.

15. An earpiece as set forth in claim 14 wherein the hole or opening at the second end of the housing comprises a hole, further wherein the cap unit has a hole for being brought into coincident relationship with the hole at the second end of the housing.

16. An earpiece as set forth in claim 14 wherein a slot is provided at the second end of the second housing and a nose is provided at the cap unit in such a way that the nose and the slot co-operate in such a way that the rotatability of the cap unit is limited by two positions.

17. An earpiece as set forth in claim 7 characterised in that the earpiece is in the form of an in-ear earpiece.
